(19) United States
(12) Patent Application Publication
(10) Pub. No.: US 2009/0104138 A1
(43) Pub. Date: Apr. 23, 2009

(54) Aerosol Hairspray Composition

(76) Inventors: Mitsuru Shimatani, Tochigi-ken (JP); Keiko Sugioka, Tochigi-ken (JP)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/920,802

(22) PCT Filed: May 17, 2006
(86) PCT No.: PCT/EP2006/004827

§ 371 Date: Nov. 20, 2007

(30) Foreign Application Priority Data
May 28, 2005 (EP) 05253320.5

(51) Int. Cl. A61K 8/81; A61Q 5/06

(52) U.S. Cl. 424/70.11

(57) ABSTRACT

A hair spray composition comprising at least one hair setting polymer comprising pendant carboxyl groups, a solvent for the setting polymer and a neutraliser for the polymer wherein the level of neutraliser present is such that at least 170% of the total carboxyl groups on the polymer could be neutralised.

### FIELD OF THE INVENTION

The invention relates to hairspray compositions.

### BACKGROUND AND PRIOR ART

Hairspray compositions must meet a number of functional requirements. These include good holding ability and curl retention without giving a harsh, brittle feeling to the hair.

Conventional hairspray formulations are frequently volatile aqueous solvent based and therefore form highly wetting systems for hair. Coalescence of the aerosol droplets on the hair fibre and subsequent ethanol evaporation to leave solid polymer residue leads to a network of fibre-fibre bonds.

There are, however, undesirable consequences in coating hair with polymer, manifesting in perceptions of sensory negatives such as stiffness, rigid feel, stickiness or unnatural feel. A further problem with formulations containing these polymers is that when applied to the hair they can leave unsightly white deposits.

We have now found that hair sprays comprising carboxylated hairspray polymers can give improved hold, better curl definition and few visible deposits by adding an increased level of neutraliser to compositions containing these polymers.

### SUMMARY OF THE INVENTION

Accordingly the present invention relates to a hair spray composition comprising at least one hair setting polymer comprising pendant carboxyl radicals, a solvent for the hair setting polymer and a neutraliser for the polymer wherein the level of neutraliser present is such that at least 150% of the total carboxyl radicals on the polymer could be neutralised.

The invention also relates to a method of styling hair comprising the step of applying to the hair a composition as described in the preceding paragraph.

A further aspect of this invention is the use in a hair styling composition of a hair setting polymer comprising pendant carboxyl groups and a neutraliser for the polymer; the level of neutraliser being such that at least 170% of the total carboxyl groups on the polymer could be neutralised.

### DETAILED DESCRIPTION OF THE INVENTION

### Film-Forming Hairspray Polymer

The hairspray polymers employed in compositions of the present invention should be capable of forming a film and holding the hair of the user in place after evaporation of the volatile components of the hairspray composition.

To provide optimum sprayability, the polymers employed in hairspray compositions typically range in number average molecular weight of from 5,000 to 100,000 with 10,000 to 50,000 being more preferred.

The amount of the polymer preferably ranges from 0.1 to 12% of the total composition, more preferably from 0.5 to 8 wt %, most preferably from 0.6 to 5 wt %.

Examples of anionic hairspray polymers are: acrylic copolymers, terpolymers, etc., containing acrylic acid or methacrylic acid as the anionic radical-containing moiety with other monomers such as: esters of acrylic or methacrylic acid with one or more saturated alcohols having from 1 to 22 carbon atoms (such as methyl methacrylate, ethyl acrylate, ethyl methacrylate, n-butyl acrylate, t-butyl acrylate, t-butyl methacrylate, n-butyl methacrylate, n-hexyl acrylate, n-octyl acrylate, lauryl methacrylate and behenyl acrylate); esters of glycols having from 1 to 6 carbon atoms (such as hydroxypropyl methacrylate and hydroxyethyl acrylate); styrene; vinyl caprolactam; vinyl acetate, acrylamide; alkyl acrylamides and methacrylamides having 1 to 8 carbon atoms in the alkyl group (such as methacrylamide, t-butyl acrylamide and n-octyl acrylamide), and other compatible unsaturated monomers. The polymer may also contain grafted silicone, such as polydimethylsiloxane.

One specific example of a suitable anionic hairspray polymer is the emulsion polymerised terpolymer of methacrylic acid, n-butyl acrylate and ethyl acrylate (e.g. in a weight percent ratio of 31:42:27, respectively)

Another specific example is Ultrahold® 8 (CTFA-Cosmetic, Toiletries and Fragrance Association designation of Acrylate/Acrylamide Copolymer).

Other suitable anionic hairspray polymers include carboxylated polyurethanes. Carboxylated polyurethane polymers are linear, hydroxyl-terminated copolymers having pendant carboxyl groups. They may be ethoxylated and/or propoxylated at least at one terminal end. The carboxyl group can be a carboxylic acid group or an ester group, wherein the alkyl moiety of the ester group contains one to three carbon atoms. The carboxylated polyurethane polymer can also be a copolymer of polyvinylpyrrolidone and a polyurethane, having a CTFA designation PVP/polycarbaryl polyglycol ester. Suitable carboxylated polyurethane polymers are disclosed in EP 0 619 111 A1 and U.S. Pat. No. 5,000,955. Other suitable hydrophilic polyurethanes are disclosed in U.S. Pat. Nos. 3,822,238; 4,156,066; 4,156,067; 4,255,550; and 4,743,673.

Amphoteric polymers are particularly preferred especially those which can contain cationic groups derived from monomers such as t-butyl aminoethyl methacrylate in combination with carboxyl groups derived from monomers such as acrylic acid or methacrylic acid can also be used in the present invention. One preferred example of an amphoteric hairspray polymer is Amphomer® (octylacrylamide/acrylates/butylaminoethyl methacrylate copolymer) sold by the National Starch and Chemical Corporation.

Particularly preferred are hairspray in which the hair setting polymer is an acrylate based copolymer.

Compositions comprising blends of polymers are advantageous, in particular blends which comprise at last two polymers and where a second polymer also comprises a carboxy groups.

Examples of suitable neutralising agents include 2-amino-2-methyl-1,3-propanediol (AMPD); 2-amino-2-ethyl-1,3-propanediol (AEPD); 2-amino-2-methyl-1-propanol (AMP); 2-amino-1-butanol (AB); monoethanolamine (MEA); diethanolamine (DEA); triethanolamine (TEA); monoisopropanolamine (MIPA); diisopropanol-amine (DIPA); triisopropanolamine (TIPA); and dimethyl stearamine (DMS). A long chain amine neutralising agent such as lauramidopropyl dimethylamine may be employed, as is described in U.S. Pat. No. 4,874,604. Mixtures of any of the above neutralising agents may be used. Amounts of the neutralising agents will range from about 0.001 to about 10% by weight of the total composition.

In terms of neutralisaton of the carboxy groups of the polymers present, the level of neutralising agent should be such that at least 170% of the total carboxyl groups could be neutralised, preferably 200% of the total carboxyl groups could be neutralised, more preferably 210% of the total carboxyl groups could be neutralised.

Nonionic hair styling polymers may also be present, suitable nonionic polymers are homopolymers of N-vinylpyrrolidone and copolymers of N-vinylpyrrolidone with compatible nonionic monomers such as vinyl acetate. Nonionic polymers containing N-vinylpyrrolidone in various weight average molecular weights are available commercially from ISP Corporation—specific examples of such materials are homopolymers of N-vinylpyrrolidone having an average molecular weight of about 630,000 sold under the name PVP K-90 and are homopolymers of N-vinylpyrrolidone having an average molecular weight of about 1,000,000 sold under the name of PVP K-120.

### Propellant

The hair sprays of the invention can utilise any of the conventional propellants to deliver the material as a spray. Examples of suitable propellants include materials such as trichlorofluoromethane, dichlorodifluoromethane, difluoroethane, dimethylether, propane, n-butane or isobutane. Hydrocarbon based propellants, especially LPG, are preferred.

The level of propellant can be adjusted as desired but is generally from about 3% to about 50% by weight based on total weight, preferably from 5 to 45%, optimally from 25 to 45% by weight of the total composition.

Suitable spray containers are well known in the art and include conventional, non-aerosol pump sprays i.e., “atomisers”, aerosol containers or cans having a propellant, as described above, and also pump aerosol containers utilising compressed air as the propellant.

### Autophobic Hairspray Additive

In some instances an autophobic hairspray additive may be present. An autophobic hairspray additive is a surfactant or polymer which imparts autophobic behaviour to the hairspray composition. Suitable autophobic hairspray additives include the following:

alkyl-pendant silicone copolyols as described in U.S. Pat. No. 4,871,529. A preferred example is the ethoxylated dimethicone copolyol SILWET® L-7602, ex OSi Specialities.

Dimethicone copolyols referred as “ABA” type copolymers, due to the presence of alternating polyalkylene oxide and silicone blocks.

Polydimethicone copolyols referred as (AB)n type copolymers.

Alkyl ethoxylates such as GENAPOL® C-250, (ex Hoechst Celanese), which is coconut fatty alcohol (C8-C18, mainly C12-C14) ethoxylated with 25 moles of ethylene oxide, and DOBANOL® 91-5 (ex Shell), which is C9-C11 alcohol ethoxylated with 5 moles of ethylene oxide;

### Optional Components

A preferred optional component in hairsprays of the invention is a conditioning agent selected from volatile and non-volatile silicone fluids. Volatile silicone fluids are preferably oils chosen from cyclic or linear polydimethyl siloxanes containing from 3 to 9, preferably from 4 to 5 silicon atoms.

Cyclomethicone is the most preferred cyclic volatile silicone. Linear volatile silicone oils generally have viscosities less than about 5 centistokes at 25° C. while cyclic fluids typically have viscosities of less than about 10 centistokes.

Non-volatile silicone oils useful for the present invention include polyalkyl siloxanes, polyalkylaryl siloxanes and polyether siloxane copolymers. Non-volatile polyalkyl siloxanes useful herein include, for example, polydimethyl siloxanes with viscosities of from 5 to 100,000 centistokes at 25° C. Among the preferred non-volatile silicones are the polydimethyl siloxanes having viscosities from 10 to 400 centistokes at 25° C. These siloxanes are available, for example, from the General Electric Company as SF 1075 methyl phenyl fluid or from Dow Corning as 556 Cosmetic Grade Fluid.

The non-volatile polyalkylaryl siloxane fluids that may be used include, for example, polymethylphenylsiloxanes having viscosities of about 15 to 30,000 centistokes at 25° C.

Also includable are minor amounts of other ingredients commonly found in hair care compositions, such as antifoam agents, antioxidants, proteins, preservatives, keratin amino acids, UV inhibitors, fragrances, coloring agents, buffering agents, polyols, and other moisturizing agents. Generally these optional ingredients are included individually at a level of up to about 5% by weight of the total composition.

Preferably, compositions of this invention also contain adjuvants suitable for hair care. Generally such ingredients are included individually at a level of up to 2%, preferably up to 1%, by weight of the total composition.

Among suitable hair care adjuvants, are:
(i) natural hair root nutrients, such as amino acids and sugars. Examples of suitable amino acids include arginine, cysteine, glutamine, glutamic acid, isoleucine, leucine, methionine, serine and valine, and/or precursors and derivatives thereof. The amino acids may be added singly, in mixtures, or in the form of peptides, e.g. di- and tripeptides. The amino acids may also be added in the form of a protein hydrolysate, such as a keratin or collagen hydrolysate. Suitable sugars are glucose, dextrose and fructose. These may be added singly or in the form of, e.g. fruit extracts. A particularly preferred combination of natural hair root nutrients for inclusion in compositions of the invention is isoleucine and glucose. A particularly preferred amino acid nutrient is arginine.
(ii) hair fibre benefit agents. Examples are:
   ceramides, for moisturising the fibre and maintaining cuticle integrity. Ceramides are available by extraction from natural sources, or as synthetic ceramides and pseudoceramides. A preferred ceramide is Ceramide II, ex Quest. Mixtures of ceramides may also be suitable, such as Ceramides LS, ex Laboratoires Serobiologiques.

The invention will now be further illustrated by the following, non-limiting Examples:

Examples of the invention are illustrated by a number, comparative Examples are illustrated by a letter.

| | Example A | Example B | Example 1 |
|---|---|---|---|
| AMPHOMER 28-4910 | 2.5 | 2.5 | 2.5 |
| RESYN 28-2930 | 0.5 | 0.5 | 0.5 |
| AMP | 0.47 | 0.81 | 1.08 |
| SILWET 55-2801 | 0.1 | 0.1 | 0.1 |
| Ethanol | 66.43 | 66.09 | 65.82 |
| LPG 0.29 Pa 30 | 30 | 30 | |
| Total | 100 | 100 | 100 |
| Neut. % in total | 97% | 168% | 225% |
| Curl retention % | 44.7 | 48.5 | 49.6 |
| (30 C., 90% RH, 60 min) | | | |

Amphomer 28-4910: typical acid value = 1.9
Resyn 28-2930: typical acid value = 1.3
Silwet 22-2801: Dimethicone copolyol

It is thus demonstrated that Examples according to the invention exhibit better than the comparative Example.

1. A hair spray composition comprising at least one hair setting polymer comprising pendant carboxyl groups, a solvent for the setting polymer and a neutraliser for the polymer wherein the level of neutraliser present is such that at least 170% of the total carboxyl groups on the polymer could be neutralised.

2. A hair spray composition according to claim 1 wherein the level of the level of neutraliser present is such that at least 200% of the total carboxyl groups on the polymer could be neutralised.

3. A hairspray composition according to claim 1, in which the hair setting polymer is an acrylate based copolymer

4. A hairspray composition according to claim 1, in which the hair setting polymer is an amphoteric copolymer.

5. A hairspray composition according to claim 1 in which the hair setting polymer comprises octylacrylamide/acrylates/butylaminoethyl methacrylate copolymer.

6. A hairspray composition according to claim 1 which further comprises a second polymer.

7. A hairspray composition according to claim 6 in which the second polymer comprises a carboxy groups.

8. A hairspray composition according to claim 1 in which the neutraliser is an aminoalkyl chain alcohol, having an alkyl chain length of from 1 to 8 carbon atoms.

9. A hairspray composition according to claim 8 in which the neutraliser is aminomethyl propanol.

10. A hairspray composition according to claim 1 in which the level of hair setting polymer is from 0.5 to 8 wt % of the total composition.

11. A method of styling hair comprising the step of applying to the hair a composition described in claim 1.

12. The use in a hair styling composition of a hair setting polymer comprising pendant carboxyl groups and a neutraliser for the polymer; the level of neutraliser being such that at least 150% of the total carboxyl groups on the polymer could be neutralised.
